(19) United States
(12) Patent Application Publication
(10) Pub. No.: US 2003/0208133 A1
(43) Pub. Date: Nov. 6, 2003

(54) Breath ketone analyzer

(76) Inventor: James R Mault, Evergreen, CO (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/297,794

(22) PCT Filed: Jun. 6, 2001
(86) PCT No.: PCT/US01/18263

(51) Int. Cl. A61B 5/08

(52) U.S. Cl. 600/532; 600/531

(57) ABSTRACT

A respiratory analyzer for a person comprises a flow path through which the person breathes; a metabolic rate meter, providing a metabolic rate for the person; a ketone sensor, providing a ketone signal related to the concentration of respiratory components correlated with a level of ketone bodies in exhalations of the person; a display; and an electronic circuit, receiving the ketone signal and the metabolic data, and providing a visual indication of the metabolic rate and the ketone signal to the person on the display. The respiratory analyzer can be used in an improved exercise management program for the person.

### FIELD OF THE INVENTION

This invention relates to the respiration analysis of a mammal, in particular to the detection of ketones in the exhaled breath of a person, and further relates to exercise monitoring and diet management.

### BACKGROUND OF THE INVENTION

The metabolism of fat, in particular the breakdown of triglycerides, leads to the accumulation of ketone bodies in the blood. These ketone bodies comprise acetone, acetoacetic acid and beta-hydroxybutyric acid. (In the blood, acetone exists in the form of acetoacetate). The concentration of acetone in exhaled breath is well correlated with the concentration of acetone in the blood. Hence, breath analysis provides valuable information about the physiological status and metabolic processes present in a person.

During periods of restricted calorie input, the concentration of ketone bodies and fatty acids increase, whereas the concentration of glucose will fall. Hence, the detection of ketone bodies in the blood or urine, or their manifestation in the exhaled breath of a person, is indicative of successful adherence to a weight control program.

In a weight control program, a person will often attempt to achieve a body weight loss through restricted calorie intake. However, weight loss can arise through a variety of mechanisms, such as fat loss, muscle loss, and water loss. Conventional weight control programs often neglect the actual mechanism by which weight is lost.

In U.S. Pat. No. 4,970,172, Kundu describes a method of measuring acetone concentration in the breath, by extracting a sample volume of breath, and allowing acetone in the breath to interact with matrix materials containing a nitroprusside salt and an amine. However, sampling methods fail to provide a real-time determination of breath ketone level.

In U.S. patent application Ser. No. 09/630,398 and international application WO01/8554, Mault et al. describe an indirect calorimeter comprising an oxygen sensor and an ultrasonic flow meter. However, this device cannot detect aldehydes and ketones in exhaled breath.

Other ketone detection methods are known in the art, for example as described by Kundu in U.S. Pat. Nos. 5,174,959, 5,071,769, 4,970,172, 4,931,404; and U.S. Pat. No. 5,834,626 to De Castro et al. However, there is no disclosure in these patents of a device for real-time monitoring of ketone levels by breath analysis.

During exercises of escalating intensity, the metabolism of fat causes ketone levels in the breath to increase.

During a restricted calorie diet, the rate of fat loss is correlated with breath acetone concentration, as disclosed by Kundu in U.S. Pat. No. 4,970,172. Hence, monitoring of acetone levels in the breath can be used to provide valuable information on exercise programs and weight loss programs.

### SUMMARY OF THE INVENTION

Weight control is an important goal of a large proportion of the U.S. population. Conventional weight control programs typically allow a restricted range of caloric intake per day, with some allowance made for activity levels. However, even though caloric intake is monitored with some precision, the effects of physical activity are not measured in a quantitative way. Physical activity is an important component of weight control programs for several reasons. It can be used to reduce the body fat proportion of a person. It can help reduce the fall in resting metabolic rate of a person on a restricted caloric intake. Activity is initially fueled by blood sugar, but after a sustained period of activity a person will start to metabolize fat. Few people on weight control programs are aware of how much exercise is required to start the fat metabolizing process, and they may not be fully aware of the beneficial effects of activity on their resting metabolic rate.

We will describe apparatus for the measurement of breath ketones, and describe an improved weight loss program which uses such apparatus. We will also describe a diet and exercise control program for people suffering from diabetes. The ketone is usually assumed to be acetone. Aldehydes such as acetaldehyde may also be detected by the methods described below.

The inventor, James R. Mault, has described an indirect calorimeter referred to as a Gas Exchange Monitor (GEM) used to measure the oxygen consumption of a person and hence their metabolic rate. The GEM comprises a bi-directional ultrasonic flow-meter and an oxygen sensor which uses the fluorescence quenching of a film by oxygen molecules. Resting metabolic rate is calculated from the measured oxygen consumption rate. The GEM has a coaxial flow geometry which enables ketone sensors to be incorporated into the device, in addition to the oxygen and flow sensors. Below, modifications to the GEM allowing ketone detection are described.

Resting metabolic rate can also be estimated from the Harris-Benedict equation, as discussed by Karkanen in U.S. Pat. No. 5,839,901, and using metabolic rate meters comprising gas sampling techniques and differential pressure based flow rate sensors, for example as disclosed by Acorn in U.S. Pat. No. 5,705,735.

In some cases, it can be useful to monitor the composition of inhaled gases, for example when administering gases to the patient such as anesthetics, nitric oxide, medications, and other treatments, monitoring pollutants or environmental effects, for a person respiring with the assistance of a ventilator, or for persons using breathing apparatus. For convenience, the analysis of exhaled gases will be discussed, though the embodiments described can also be used for analysis of inhaled gases.

An indirect calorimeter can be advantageously modified to detect exhaled breath components. For example, a radiation emitter can be used to emit radiation, the radiation being directed along a flow path for exhaled air, and being detected by a radiation detector. Absorption of the radiation will result in a decrease in the detector signal from the detector. The sensitivity can be improved using optical filters to remove extraneous radiation. Time modulation of emission can also be used to improve sensitivity e.g. by phase locking methods. The radiation emitter and radiation detector can built into the body of an indirect calorimeter, having a flow path carrying exhaled air. The flow path can forms part of a removable portion of an indirect calorimeter, which may be disposed of or sterilized between use of the respiratory analyzer. The respiratory analyzer can further comprise ultrasonic flow sensors, ultrasonic gas density sensors (from which carbon dioxide concentration in the exhalation can be determined), an oxygen sensor, a carbon dioxide sensor, and other gas component sensors.

In a preferred embodiment, the radiation emitter is a source of IR radiation at a wavelength which will be absorbed by the molecules to be detected. For example, acetone has a strong IR absorption near 1700 cm⁻¹ due to carbonyl group stretching vibrations, and medium strength absorption due to carbon-hydrogen bond vibrations. In a preferred embodiment, carbonyl stretching vibrations are detected by IR absorption. Overtones of the carbonyl stretching vibration, in which higher vibrational states are excited, may be detected in the near-IR, for example using a semiconductor laser and a near-IR detector. Aldehyde molecules such as acetaldehyde can also be detected using carbonyl absorption methods.

The path-length of the emitted radiation through the respired air can be increased by reflecting the radiation so as to make a number of passes through the flow path. The path length can be further increased by multiple reflections.

The radiation emitter may be a thermal emitter, light-emitting diode (LED), laser, or other luminescent source. LEDs and lasers may be semiconductor, polymer, or other organic material. In a preferred embodiment, a semiconductor IR emitter is used. Time-dependent modulation of the emitted signal can be used in lower-noise detection schemes (e.g. using phase locking of emitted and detected radiation). Lenses and/or mirrors may be used to focus or steer the beam. In other embodiments, a radiation emitter and/or radiation detector can be provided by and external device, with radiation channeled by waveguides, optical fibers, and the like.

The radiation detector may be a bolometer, photoelectric device, photoconductor, photodiode, etc. In a preferred embodiment, the detector is a semiconductor IR detector., e.g. a photodiode, photoconductive, photoelectric, or quantum well detector using such materials as silicon, cadmium selenide, cadmium telluride, indium gallium arsenide, and the like.

A mirror can comprise a metal film, semiconductor film, dielectric film, multilayer structure, etc., possibly with a protective coating. In a preferred embodiment, a gold film is used. An ultrasonic flow sensor can be used to detect the onset of exhalation. Detection may be electronically delayed by a specified time period in order to ensure deep lung alveolar breath is sampled.

Ketone detection can also be achieved using a hand-held respiratory analyzer used in accompaniment to an indirect calorimeter in an improved weight control program. A person holds the analyzer to their mouth, and breathes through a mouthpiece. Exhaled air is conveyed along a flow tube. The exhaled air may be dried by conventional means, e.g. using silica gel. Preferably, the drying process should not remove a substantial proportions of the gas component of interest from the expired air. Volatile organic compounds such as acetone can be condensed as a film on a cooled surface, and detected by spectroscopy (such as attenuated total reflection IR spectroscopy), colorimetry, and the like. Selectively permeable membranes may also be used to allow nitrogen, oxygen, and possibly carbon dioxide to exit a detector device, while concentrating volatile organics such as ketones for detection by any appropriate method.

Exhaled air vented from a respiratory analyzer can be further analyzed, for example by routing the exhaled air to an analytical device such as a mass spectrometer, chromatography device, calorimeter, or other instrument. For example, ketones and other volatile organic compounds in exhaled gases can be detected by gas chromatography. The exhaled air is passed through a flame and combustion reactions are detected using characteristic optical emission and/or absorption lines. Oxygen, carbon dioxide, nitrogen, and rare gases are not combusted by a flame, but in the breath such as ketones are combusted. In U.S. Pat. No. 4,114,422, Hutson describes a hydrogen flame ionization scheme to detect acetone in the breath, which can be advantageously combined with an indirect calorimeter.

A respiratory analyzer, such as an indirect calorimeter, can be advantageously adapted to detect respiratory components by chemical methods. For example, a disposable flow path element of an indirect calorimeter, such as a removable element comprising a tube, a mouthpiece, or an exhaust vent, can have a film disposed on a surface exposed to exhaled gases. The film changes color, or provides some other visual indication, of a respiratory component in the exhaled gases. The film can be processed after removal from the indirect calorimeter to enhance the indication. In U.S. Pat. No. 4,758,521, Kundu describes adsorption of ketones onto solid pellets, and chemical detection using a nitroprusside salt in one solid matrix, with an amine coupled to a second solid matrix. Chemical detection methods such as this may be incorporated into the disposable part of the GEM (gas exchange monitor) described by James R. Mault. Colorimetry may be used to detect the onset of significant levels of fat burning by the person's metabolic processes; such threshold-type detection does not need an updateable real-time ketone concentration reading.

Data may be transferred from the ketone sensor to other devices such as a portable computer, personal digital assistant (PDA), interactive television component (e.g. set-top box, web-TV box, cable box, satellite box, etc.), desk-top computer, wireless phone, etc. via Bluetooth protocol radio communication, IR communication, transferable memory sticks, wires, or other electromagnetic/electrical methods. Data may also be transferred to a remote computer via a communications network such as the Internet. In a preferred embodiment, data is transferred to a PDA using Bluetooth radio communication.

In another embodiment, a fluorescence quenching ketone detector is used. A fluorescent film is illuminated with radiation, causing it to fluoresce. The film has a surface layer which specifically adsorbs or otherwise interacts with ketones, causing fluorescence quenching of the film, and hence measurement of ketone concentrations in the gases passing over the sensor. This approach may be used by providing a fluorescence quenching ketone detector in the breath path of the calorimeter or in a breath path in a stand alone ketone detector. The fluorescence quenching ketone detector allows real time analysis of ketone concentrations.

The following example illustrates how breath ketone measurements can be used in an improved weight loss program involving an exercise component. A person is equipped with an activity sensor (e.g. pedometer, accelerometer) and starts an activity routine (e.g. running on the spot). A Gas Exchange Monitor (GEM) with additional ketone sensing capability is used to monitor the person's oxygen intake rate and hence metabolic rate; and also to detect the attainment of a certain acetone level in the person's breath, indicating the onset of fat catabolism. The data is transferred to a portable electronic device, such as a personal digital assistant (PDA). Data transfer to the PDA may be using IR communication, Bluetooth protocol wireless communication, or through the transfer of a memory stick (such as those manufactured by Sony or SanDisk). The data can be used to create a model of the person's physiological response to exercise.

During a daily exercise routine, a signal from the activity sensor is transferred to the PDA, preferably using the Bluetooth protocol. The PDA is then used to provide quantitative feedback to the person on the benefits of the exercise. For example, the PDA may be used to indicate the calories burned, the time the exercise must continue for the onset of fat burning, or an estimate of fat grams burned. This level of feedback is a great improvement over previous weight control/exercise programs, and a very powerful motivational factor for the person to continue with the exercise.

The following example illustrates a diet and exercise control program for a person suffering from diabetes. The person carries a personal digital assistant (PDA), and has a glucose sensor transmitting blood glucose levels to the PDA using a wireless transmission protocol such as Bluetooth. Dietary intake is entered into the PDA. The PDA is used to track dietary intake and blood sugar levels, estimate possible future deviations of blood sugar from an acceptable range, and provide warnings and advice to the person. Indirect calorimetry is used to determine the metabolic rate of the person. An activity sensor is used to provide a signal correlated with physical activity. These data are transmitted to the PDA, preferably using Bluetooth. Breath ketone sensing is used to detect the onset of the dangerous condition of ketoacidosis.

A system for warning a person of the onset of ketoacidosis comprises a portable computing device carried by the person, a blood glucose sensor, and a respiratory analyzer (which device functions of indirect calorimeter and respired volatile organics detector, in two way communication using wireless communication. Data may also be transferred to or from any device using non-volatile memory cards, or via a wire.

The PDA and respiratory analyzer may be combined into a portable unitary device, or the respiratory sensors may be attached to the PDA for use. Also, the ketone sensing device may be combined or be separate from the calorimeter.

The following example relates to exercise management. A person exercising carries a portable ketone analyzer that includes a tube that is breathed through and a fluorescence quench ketone detector disposed on one wall of the tube. The device may be small, such as the size of a lighter. The exerciser may periodically blow through the device to determine whether they are burning fat. Alternatively, the device may prompt the user to periodically blow, or may signal that analysis is required after a certain period of time has passed. Also, a separate exercise monitor may wirelessly signal the analyzer that a breath should be analyzed after a certain set of conditions are met. The analyzer may wirelessly communicate the results back the an exercise monitor, may give a confirmation of results such as by a chime indicating fat burning, or may store the results versus time onto a non-volatile memory device. The memory device may later be removed from the analyzer and inserted in another computing device for retrieval of the data.

Hence, a method for encouraging exercise in a person comprises: monitoring a metabolic rate of a person during an exercise, and hence correlating the exercise with metabolic rate; detecting the presence of organic compounds in the breath of the person, indicative of fat metabolizing processes in the person, and hence determining the effect of exercise on fat burning; providing feedback to the person during future repetition of the exercise, in terms of the effect of the exercise on metabolic rate and fat burning

whereby the person is encouraged to continue exercising by the provision of the feedback.

A device for the detection of a component of an exhaled breath of a person comprises: a flow path through which the exhaled breath passes, a radiation emitter producing radiation, the radiation passing through the flow path; and a radiation detector, detecting the radiation after the radiation has passed through the flow path. Hence, the organic compound can detected by absorption of the radiation by the component. In one embodiment, the radiation emitter is an IR emitter, the radiation detector is an IR detector, and the flow tube has a coaxial geometry. In another embodiment, the detector can detect fluorescence produced by the component through interaction with the radiation.

Embodiments of the present invention can be used to detect numerous volatile organic compounds in the breath, which include ketones such as acetone, aldehydes such as acetaldehyde, hydrocarbons including alkanes such as pentane, alkenes, and fatty acids, and other compounds for example as disclosed in U.S. Pat. No. 5,996,586 to Phillips, and in U.S. Prov. App. No. 60/228,680. Embodiments of the present invention can further be used to detect nitric oxide, ammonia, carbon monoxide, carbon dioxide, and other components of exhaled breath. Respiration components produced by certain bacteria within the mouth, stomach, and intestinal tract can also be detected using embodiments of the present invention.

The mouthpiece of an indirect calorimeter (or other respiratory analyzer) can contain a film, patch, test strip, or other structure sensitive to a respiration component. This can be used to indicate that a mouthpiece has been previously used. A test strip exposed to exhaled air can be used to provide calorimetric indication of breath components. A person can insert a test strip, which can be moist, into a suitably adapted mouthpiece of an indirect calorimeter before use, for example securing the strip on the inside surface of a respiratory connector. The person then breathes through the indirect calorimeter for several minutes to measure their metabolic rate. After this period, the test strip is removed and examined or otherwise analyzed for indication of the respiration component. For example, ketones can be detected using a test strip containing nitroprusside salts. Test strips may be moistened with water, or infused with other hydrophobic (or hydrophilic) solvents. For example, an oily film (or test strip) may be preferred for selective absorption of organic components of the breath, for example for colorimetric detection.

A respiratory analyzer according to the present invention can be combined with gas flow sensors so as have the capabilities of a spirometer. The improved spirometer is useful for detecting respiratory components such as nitric oxide diagnostic of asthma and other respiratory tract inflammations. The combination of respiratory component analysis and flow rate analysis is helpful in diagnosing respiration disorders.

Certain persons desire a diet low in carbohydrates and high in protein. A respiratory analyzer according to the present invention can be used to detect respiration components indicative of success in following such a diet.

Hence, an improved respiratory analyzer for a person, comprises: a flow path, through which the person breathes; a metabolic rate meter, providing metabolic data correlated with the metabolic rate of the person; a ketone sensor, providing a ketone signal correlated with a concentration of respiratory components in exhalations of the person, wherein the respiratory components are correlated with a level of ketone bodies in the blood of the person; a display; and an electronic circuit, receiving the ketone signal and the metabolic data, and providing a visual indication of the metabolic rate and the ketone signal on the display. The metabolic rate meter can comprise a pair of ultrasonic transducers, for example using the density of exhaled air to determine oxygen and carbon dioxide concentrations in exhaled air, as described in Int. App. WO00/7498. The metabolic rate meter may comprise a flow rate sensor, and an oxygen sensor and/or a carbon dioxide sensor, for example as discussed in U.S. patent application Ser. No. 09/630,398. Embodiments of the ketone sensor are discussed in detail below. The ketone sensor can, for example, comprise a radiation emitter and a radiation detector, the radiation emitted by the radiation emitter passing through a part of the flow path. The ketone sensor can comprise a fluorescence film, the fluorescence intensity of the fluorescence film being correlated with ketone concentrations in the flow path through a quenching mechanism.

A respiratory analyzer can comprise a flow path operable to receive and pass exhaled gases, the flow path having a first end in fluid communication with a respiratory connector and a second end in fluid communication with a source and sink for respiratory gases, the respiratory connector configured to be supported in contact with the subject so as to pass exhaled gases as the subject breathes, the flow path comprising a flow tube through which the exhaled gases pass, and a chamber disposed between the flow tube and the first end, the chamber being a concentric chamber surrounding one end of the flow tube, for example as disclosed in U.S. patent application Ser. No. 09/630,398, and further comprises a ketone sensor, providing a signal correlated with the presence or concentration of at least one exhaled breath component correlated with ketone body levels in the blood of the person A flow rate sensor, for example a pair of ultrasonic transducers, can be used to determine respired volumes, volumes of breath components, and the start and stop of inhalations and exhalations.

An improved exercise management program for a person comprises: providing an activity monitor to the person, the activity monitor providing an activity signal correlated with the physical activity level of the person; providing a metabolic rate meter to the person, the metabolic rate meter providing a metabolic rate data for the person; and providing a respiratory analyzer having a ketone sensor to the person, the ketone sensor providing a ketone signal correlated with ketone levels in the person's exhalations. The person perform an activity, while monitoring the activity signal from the activity monitor, the metabolic rate data from the metabolic rate meter, and the ketone signal from the ketone sensor. The activity signal is then correlated with the metabolic rate signal and the ketone signal; so that after this correlation step the activity signal can then be used to determine a metabolic rate and an estimate of fat burning for the person during activities and exercise programs. The metabolic rate meter and the respiratory analyzer having the ketone sensor can form a unitary device.

In this specification, the terms ketone and ketones are used in relation to respiratory analysis to refer to respiratory components correlated with the levels of ketone bodies in the blood. These respiratory components include acetone, acetaldehyde, and beta-hydroxybutyric acid. Hence, a ketone sensor may refer to an acetone sensor, an acetaldehyde sensor, or a beta-hydroxybutyric sensor, or a sensor responsive to the presence of one or more respiratory components correlated with ketone body concentration in the blood of a person.

The contents of the following are incorporated herein by reference: U.S. patent application Ser. No. 09/630,398 and Int. App. Nos. WO01/28495, WO01/28416, WO01/26547, WO01/26535, WO01/8554, and WO00/7498 to Mault et al.; U.S. Provisional App. Nos. 60/210,034 (filed Jun. 7, 2000), 60/225,101 (filed Aug. 14, 2000), 60/225,454 (filed Aug. 15, 2000), 60/228,388 (filed Aug. 28, 2000), 60/228,680 (filed Aug. 29, 2000), and 60/257,138 (filed Dec. 20, 2000); U.S. Pat. No. 4,114,422 to Hutson; U.S. Pat. No. 4,758,521 to Lushbaugh et al.; U.S. Pat. Nos. 5,174,959, 5,071,769, 4,970,172, 4,931,404, all to Kundu et al.; U.S. Pat. No. 5,834,626 to De Castro et al.; U.S. Pat. No. 5,996,586 to Phillips; U.S. Pat. No. 5,705,735 to Acorn; U.S. Pat. No. 5,839,901 to Karkanen; U.S. Pat. No. 5,932,812 to Delsing; and U.S. Pat. Nos. 6,135,107, 5,836,300, 5,179,958, 5,178,155, 5,038,792, and 4,917,108, all to Mault et al.

### BRIEF DESCRIPTION OF THE DRAWINGS

FIG. 1 shows a cross-section of a respiratory analyzer according to the present invention;

FIG. 2 shows a simplified cross section of the respiratory analyzer of FIG. 1;

FIG. 3 shows a schematic of an analysis circuit for a respiratory analyzer;

FIG. 4 shows a cross section of a respiratory analyzer employing reflection;

FIG. 5 shows a cross section of a respiratory analyzer employing multiple reflections;

FIG. 6 shows a cross section of a respiratory analyzer employing ultrasonic transducers;

FIG. 7 is a flowchart for a method of respiratory analysis;

FIG. 8 is a flowchart for a method of respiratory analysis;

FIGS. 9 and 9A show an indirect calorimeter according to a co-pending application to Mault et al.;

FIG. 10 shows a cross-section of an indirect calorimeter according to a copending application to Mault et al.;

FIG. 11 illustrates an improved physical activity monitoring system;

FIG. 12A shows a schematic of an activity monitor used in the system of FIG. 11;

FIG. 12B shows a schematic of a portable computing device used in the system of FIG. 11;

FIG. 13 illustrates diet and exercise control system for a person suffering from diabetes;

FIG. 14 shows another geometry for a respiratory analyzer;

FIG. 15 shows a respiratory analyzer having a fluorescence sensor;

FIG. 16 shows a possible embodiment of a fluorescence ketone sensor;

FIG. 17 shows a respiratory analyzer having a cylindrical housing;

FIG. 18 shows a respiratory analyzer having a laser fluorescence detector;

FIG. 19 shows a respiratory analyzer having both backwards and forwards scattering/fluorescence detection;

FIG. 20 shows a respiratory analyzer with fluorescence detection;

FIG. 21 shows a respiratory analyzer with photoionization detection.

### DETAILED DESCRIPTION OF THE INVENTION

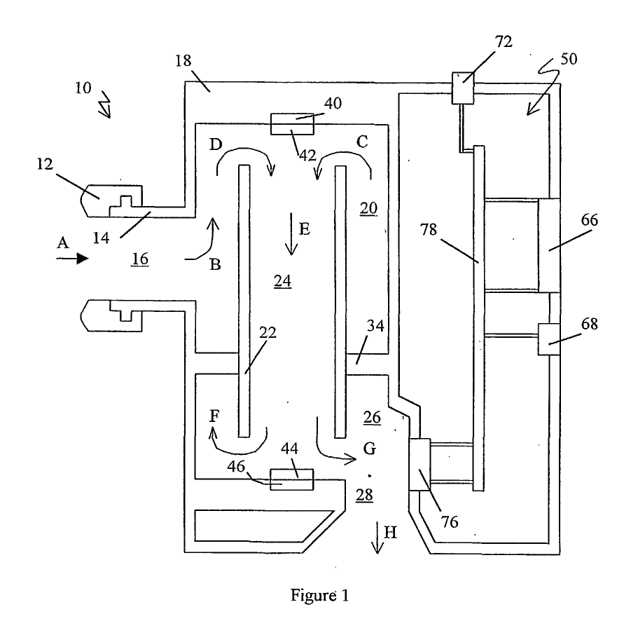

FIG. 1 shows a cross-sectional view of a breath ketone analyzer having a coaxial flow geometry. A similar flow geometry is more fully described in copending application U.S. patent application Ser. No. 09/630,398 to Mault et al., and is discussed further in relation to an embodiment of the Gas Exchange Monitor, an indirect calorimeter, below.

The analyzer 10 comprises a mouthpiece 12, inlet tube 14 surrounding an inlet path 16, and a main housing 18 surrounding a chamber 20 concentric around flow tube 22. Flow tube 22 encloses a central flow path 24. Exhaled air passes through the central flow path 24, and enters a second concentric chamber 26, with which it is fluid coupled. In other embodiments, the second concentric chamber may be omitted. Exhaled air then exits through exhaust path 28, surrounded by exhaust tube 30, and exits through outlet 32.

The lettered arrows illustrate possible exhalation flow paths through the coaxial geometry. Air enters the first coaxial chamber 20 along paths such as A and B, and enters central flow path 24 along paths such as C and D. Flow along the central flow path is illustrated by arrow E. Air exits the central flow path into second concentric chamber 24 along paths such as F and G. Air exits the device along paths such as H.

The mouthpiece and/or inlet tube can be detachable, and may comprise pathogen filters, air drying chemicals, carbon dioxide scrubbers, and other gas processing mechanisms. A partition 34 separates the first and second concentric chambers. The flow path through the device is enclosed by the inner surface of the main housing 18. A radiation emitter 40 is disposed on the inner surface of the housing, and emits radiation which is detected by detector 46. Optical filters 42 and 44, one or both of which can be omitted, are used to modify the spectral output of the emitter, and to protect the detector from extraneous light, respectively.

The analysis module 50, shown as a separate compartment from the flow path, comprises an electronic circuit, mounted on circuit board 78, which receives signals from detector 46, and is used to control (e.g. modulate) radiation emitted by radiation emitter 40. A button 72 can be pressed to initiate a breath test. An indicator light 68 can be used to show operation of the device. In FIG. 1, a fluorescence gas sensor 76 is shown mounted on the circuit board 78. This sensor can be omitted, or be used to sense a respiratory component such as oxygen or carbon dioxide. The analysis module 50 is discussed in more detail below, and may be a detachable unit.

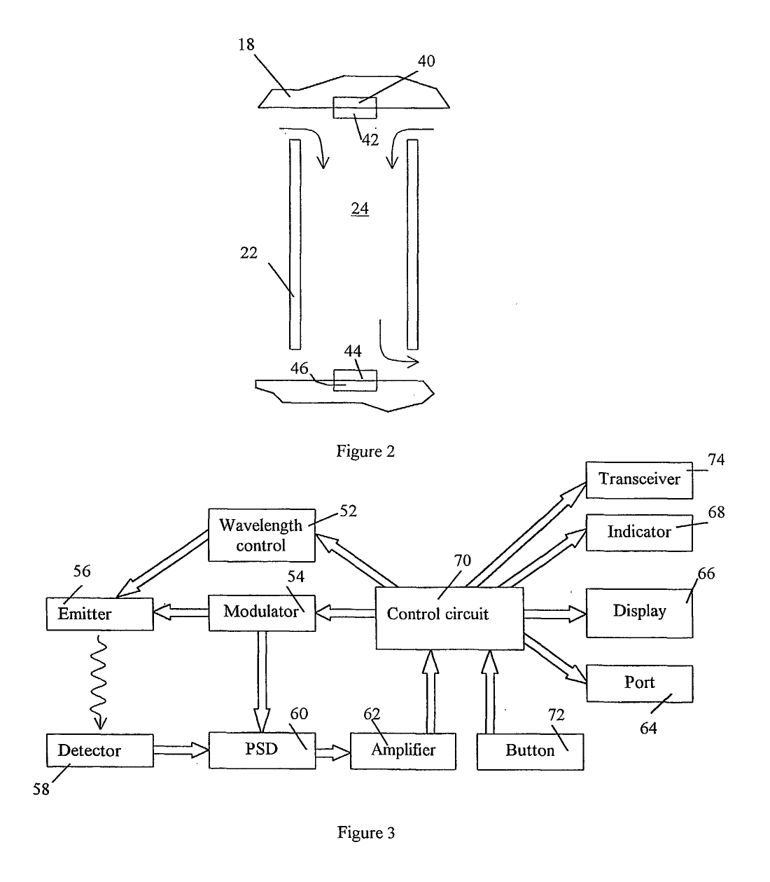

FIG. 2 shows a simplified representation of the device of FIG. 1, showing part of the housing 18, radiation emitter 40, optical filter 42, optical filter 44, radiation detector 46, flow tube 22, central flow path 24, first concentric chamber 20, and second concentric chamber 24. Other embodiments will be described relative to the simplified representation of FIG. 2, and other device components, which can be as shown in FIG. 1, will not be discussed further for convenience and clarity. In this embodiment, the radiation from the radiation emitter propagates parallel to the central flow path. In other embodiments, the radiation may propagate perpendicular, or at some oblique angle to, the central flow path.

FIG. 3 shows a schematic diagram of the electronic circuit within the analysis module 50. The analysis module comprises a processor (or other control circuit) 70, a wavelength control 52, a modulator 54, a phase sensitive detector 60, an amplifier 62, a data port 64, a display 66, an indicator light 68, a control button 72, and a transceiver 74. The modulator 54 is used to modulate output from radiation emitter 56, and wavelength control 52 is used to adjust the wavelength of the radiation emitted from the radiation emitter. The phase sensitive detector receives signals from radiation detector 58 and from modulator 54.

Control circuit 70, which can comprise a processor, provides a signal to radiation emitter 56, enabling source 56 to radiate. The signal is modulated by modulator 54, and a modulation signal is provided to phase sensitive detector circuit 60. A detector signal from detector 58 passes through the phase sensitive detector 60 to amplifier 61, and then to control circuit 70. Attenuation of radiation by an analyte between source 56 and detector 58 causes the detector signal to decrease. This decrease is interpreted by the control circuit 50 so as to provide a visual representation of analyte levels on the display 66. For example, the control circuit can comprise an analog to digital converter, and a digital value can be presented on the display.

The transceiver 74 can be used to transmit, preferably by a wireless method such as the Bluetooth protocol, measured data to another device such as a portable computing device. A cable connection to data port 64 can also be used to send data to another device.

In use, the person presses the button 72 to initiate a reading, breathes through the device, and may press the button again after the end of an exhalation. In other embodiments, to be described later, flow sensors can be used to detect the onset and cessation of an exhaled breath, and can be used to provide control signals for initiating readings.

For improved accuracy, the attenuation of radiation due to the analyte can be referenced against attenuation at another wavelength. For example, using certain semiconductor radiation emitters, the emission radiation can be changed by application of an external electric field. Hence, reference attenuation can be obtained by application of an electric field to the radiation emitter.

Alternatively, attenuation can be compared with attenuation due to an inhaled breath, as atmospheric ketone detection is negligible. Further, the device can be used to measure attenuation due to two or more components of the breath, for example ketones, carbon dioxide, and nitric oxide. A second radiation emitter and radiation detector pair can be provided so as to provide a reference channel or second component analysis.

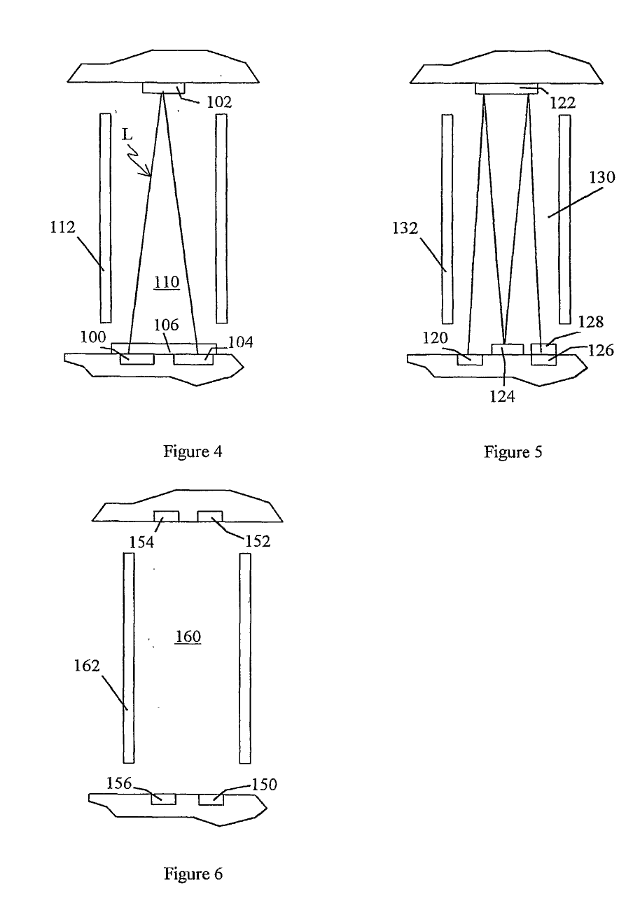

The sensitivity of detection can be improved by reflection of the radiation. FIG. 4 shows a radiation emitter 100, mirror 102, detector 104, and optical filter 106, arranged so as to measure analyte concentration in central flow path 110, bounded by flow tube 112. Radiation from radiation emitter 100, indicated as radiation beam L, is reflected by mirror 102 to radiation detector 104.

FIG. 5 shows a device having radiation emitter 120, mirrors 122 and 124, radiation detector 126, and optical filter 128. Radiation from radiation emitter 120 is reflected twice, by mirrors 122 and 124, to radiation detector 126. This allows measurement of analyte concentration in flow path 130, enclosed by flow tube 132.

Ketone Sensor with Ultrasonic Flow Sensor

FIG. 6 shows part of a ketone analysis device as described with reference to FIG. 1, further comprising a pair of ultrasonic transducers. FIG. 6 shows a device having radiation emitter 150, radiation detector 152, first ultrasonic transducer 154, second ultrasonic transducer 156, central flow path 160, and flow tube 162. Other components of the device are not shown for convenience, and can be as described in FIG. 1. Ultrasonic transducers can be piezoelectric devices, such as used by Harnoncourt as described in U.S. Pat. Nos. 5,647,370, 5,645,071, 5,503,151, and 5,419,326, incorporated herein by reference, micromachined sensors as supplied commercially by Sensant, CA, or some other transducer.

In U.S. patent application Ser. No. 09/630,398, Mault et al. describe the integration of an oxygen sensor signal with a flow rate signal so as to determine oxygen volume in exhaled air. The methods and electronic circuitry required, disclosed U.S. patent application Ser. No. 09/630,398, can be included into the analysis module 50 as shown in FIG. 1. Alternatively, a ketone sensor according to the present invention can be added to the gas exchange monitor disclosed in embodiments of U.S. patent application Ser. No. 09/630,398.

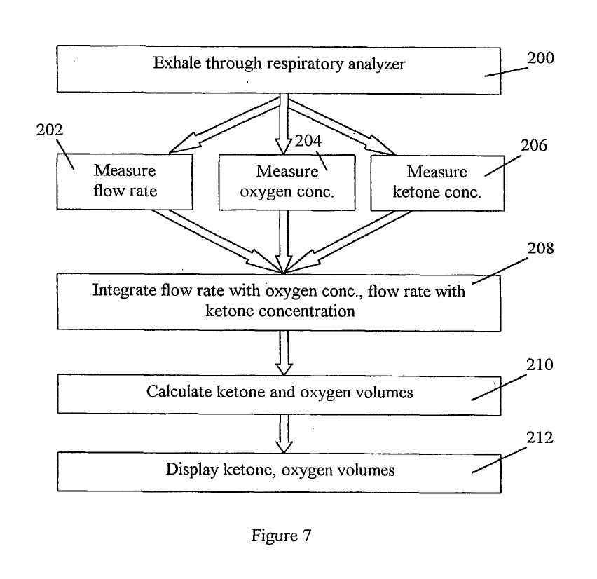

Integration of a ketone concentration signal with a flow rate signal will provide a determination of the ketone volume in the exhaled breath. FIG. 7 illustrates a flow chart representing a method of respiratory analysis. Box 200 corresponds to exhalation through the device. Box 202 corresponds to measurement of flow rate. Box 204 corresponds to measurement of oxygen concentration. Box 206 corresponds to measurement of ketone concentration. Box 208 corresponds to integration of flow rate with oxygen concentration and with ketone concentration. Box 210 corresponds to calculation of oxygen volume and ketone volume, and box 212 corresponds to the display of oxygen volume and of ketone volume to the person. In other embodiments the oxygen volume consumed, metabolic rate, and ketone concentration in exhaled breath are displayed to the person using the device.

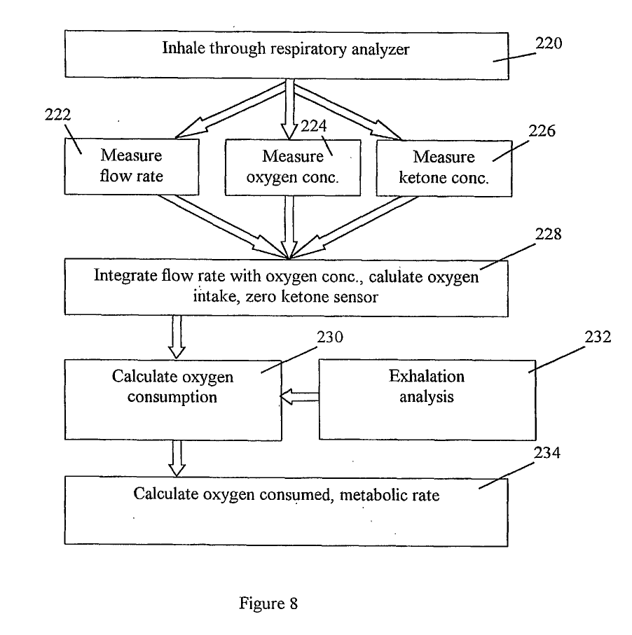

FIG. 8 illustrates a method of analyzing inhaled breath. Box 220 corresponds to a person inhaling through the device. Box 222 corresponds to measuring the flow rate, for example using a pair of ultrasonic transducers. Box 224 corresponds to the monitoring of the signal from an oxygen sensor. Box 226 corresponds to the monitoring of the signal from a ketone sensor. Box 228 corresponds to the analysis of the sensor signals. This includes the calibration of the oxygen sensor, as the concentration of oxygen in the atmosphere is known. This also includes the zeroing of the ketone sensor, as the concentration of ketones in the atmosphere is negligible. Box 230 corresponds to the calculation of oxygen consumption by the person, in view of the inhaled oxygen volume and the exhalation analysis of FIG. 7, here represented by box 232. The production of carbon dioxide by the person can also be calculated, in addition to or instead of the calculation of oxygen consumption. Box 234 corresponds to the display of oxygen consumption, metabolic rate, and ketone concentration in exhaled breath to the user.

In other embodiments, with reference to FIGS. 7 and 8 above, a signal from a carbon dioxide sensor can be monitored in place of, or in addition to, a signal from an oxygen sensor.

For exhaled breath, the temperature of the exhaled gases can be assumed to be at or close to body temperature. For inhaled breath, the ambient temperature can be used. The exhaled humidity can be assumed to be 100%. A humidity sensor can be provided in order to measure the inhaled humidity or ambient humidity. The pressure of inhaled and exhaled breaths can be assumed to be the same; however, a pressure sensor may be provided so as to convert calculated gas volumes to standard conditions. These calculations are fully described in U.S. patent application Ser. No. 09/630,398. If the device is used for exhalation analysis only, the measurement of exhaled oxygen may not be useful. The device can instead measure other diagnostic breath components, such as volatile organic compounds (VOCs), nitric oxide (NO), carbon dioxide, and other known breath components, such as discussed in U.S. Pat. No. 5,996,586 to Phillips, and in U.S. Prov. App. No. 60/228,680.

Other embodiments of the device can use ultrasonic measurements of exhaled gas density, or IR absorption measurements, so as to determine the carbon dioxide concentration in the exhaled breath. In this case, metabolic rate can be determined from the carbon dioxide production.

Gas Exchange Monitor (GEM)

FIGS. 9A and 9B illustrate a person breathing through a mask connected to an indirect calorimeter, the Gas Exchange Monitor (GEM), an indirect calorimeter developed by James R. Mault M.D. and others. Referring to FIGS. 9A and 9B, the calorimeter according to U.S. application Ser. No. 09/630,398 is generally shown at 300. The calorimeter 300 includes a body 302 and a respiratory connector, such as mask 304, extending from the body 302. In use, the body 302 is grasped in the hand of a user and the mask 304 is brought into contact with the user's face so as to surround their mouth and nose, as best shown in FIG. 9A. Optional straps 305 are also shown in FIG. 9A. With the mask 304 in contact with their face, the user breathes normally through the calorimeter 300 for a period of time. The calorimeter 300 measures a variety of factors and calculates one or more respiratory parameters, such as oxygen consumption and metabolic rate. A power button 306 is located on the top side of the calorimeter 300 and allows the user to control the calorimeter's functions. A display screen is disposed behind lens 308 on the side of the calorimeter body 302 opposite the mask 304. Test results are displayed on the display following a test. Other respiratory connectors can be used, for example a mouthpiece.

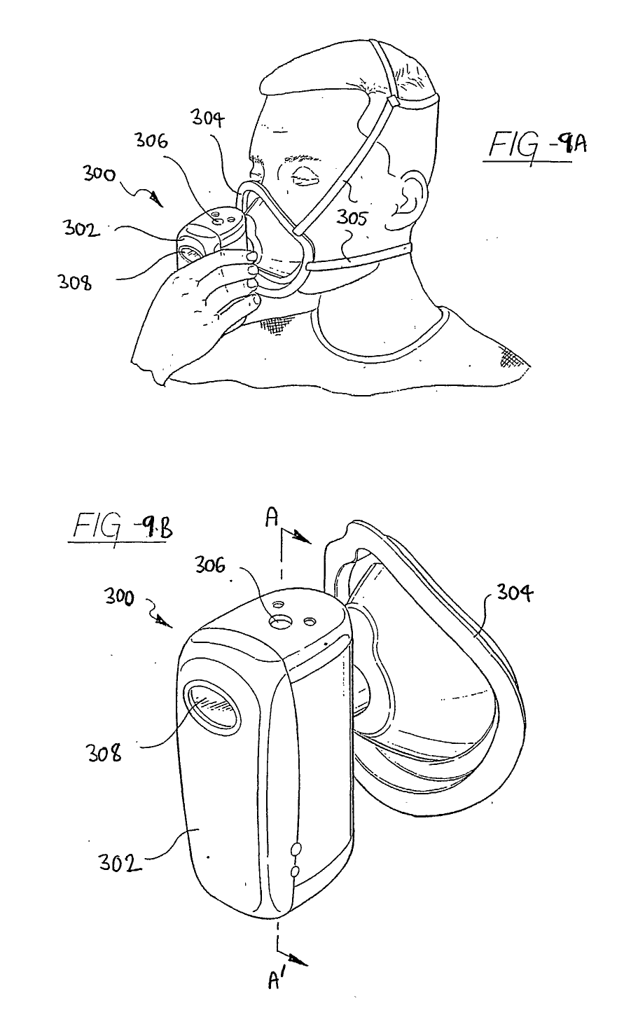

FIG. 10 shows a cross section of an indirect calorimeter, which can be used in embodiments of the present invention. The indirect calorimeter is best described in U.S. application Ser. No. 09/630,398, incorporated herein by reference. FIG. 10 shows a vertical cross section of the calorimeter 300, along section line A-A′ of FIG. 9B. The flow path for respiration gases through the calorimeter 300 is illustrated by arrows A-H. In use, when a user exhales, their exhalation passes through the mask 304, through the calorimeter 300, and out to ambient air. Upon inhalation, ambient air is drawn into and through the calorimeter and through the respiratory connector to the user.

Exhaled air passes through inlet conduit 310, and enters connected concentric chamber 312. Excess moisture in a user's exhalations tends to drop out of the exhalation flow and fall to the lower end of the concentric chamber 314. Concentric chamber 312 serves to introduce the respiration gases to the flow path 316 from all radial directions as evenly as possible. Exhaled air flows downwardly through a flow path 316 formed by the inside surface of the flow tube 318. Exhaled air enters outlet flow passage 320, via concentric chamber 322, and passes through the grill 324 to ambient air.

Flow rates through the flow path 316 are determined using a pair of ultrasonic transducers 326 and 328. An oxygen sensor 330, in contact with respiratory gas flow through opening 332, is used to measure the partial pressure of oxygen in the gas flow. Integration of oxygen concentration and flow rate allows inhaled oxygen volume and exhaled oxygen volume to be determined. The metabolic rate of the user is determined from the net oxygen consumption; the difference between inhaled and exhaled oxygen volumes. Metabolic rate is determined using either a measured or assumed respiratory quotient (the ratio of oxygen consumption to carbon dioxide production). For a user at rest, the REE (resting energy expenditure) is determined. The REE value is shown on display 309, behind window 308. Alternatively, VO₂ can be displayed, from which REE can be determined using the Weir equation, as is well known in the art.

Preferably, the indirect calorimeter used in embodiments of the present invention comprises a respiratory connector such as a mask or mouthpiece, so as to pass respiration gases as the subject breathes; a flow path between the respiratory connector and a source and sink of respiratory gases (such as the atmosphere) which receives and passes the respiration gases; a flow meter configured to generate electrical signals as a function of the instantaneous flow of respiration gases passing through the flow path, such as an ultrasonic flow meter; and a component gas concentration sensor, such as a fluorescent oxygen sensor, which generates electrical signals as a function of the instantaneous fraction of gases such as oxygen and/or carbon dioxide in the respiration gases they pass through the flow path, such as the indirect calorimeter described above. Other oxygen sensor technologies can be used, for example based on thermal, chemical, optical, surface, electrical, or magnetic effects. The user's resting metabolism can be measured at repeated time intervals using the indirect calorimeter. The user breathes a multiple of inhalations and exhalations through the indirect calorimeter, so that the inhaled air and exhaled gas passes through the indirect calorimeter, the inhaled air volume and the exhaled flow volume are integrated with the instantaneous concentration of oxygen, and so the exhaled, inhaled, and consumed oxygen are determined. The component gas concentration sensor can be omitted if the molecular mass of respired gases is determined using an ultrasound method, in which case oxygen volumes consumed can be determined using ultrasound without a component gas sensor. Other indirect calorimeters can be used in embodiments of the present invention, for example such as described in U.S. Pat. Nos. 4,917,104; 5,038,792; 5,178,155; 5,179,958; 5,836,300, and 6,135,107 all to Mault. The indirect calorimeter can also be a module which interfaces with the PDA. The display, buttons, and process capabilities of the PDA are used to operate the module, display instructions for use of the indirect calorimeter, initiate tests, and record data.

The cross-sectional area, length, and flow impedance of the flow path of an indirect calorimeter can be adjusted according to the transducers used, expected flow rates (which will be higher during exercise), desired accuracy, and other considerations.

Activity Points

A person wears breathes through an indirect calorimeter while undergoing exercise of increasing intensity. The metabolic rate of the person increases, and this increase can be measured using the indirect calorimeter. The activity energy expenditure (AEE) can then be correlated with the intensity of the exercise.

The onset of fat metabolism can be determined by the detection of ketones in the exhaled breath of the person. In order to encourage exercise, a person can receive activity points based on energy expenditure, as described in a co-pending PCT application to James R. Mault M.D., filed on May 24, 2001, incorporated herein by reference. For example, an expenditure of 100 kilocalories of energy can be designated as one point of energy expenditure.

However, exercise at intensities above which are necessary to induce fat metabolism are highly beneficial in weight loss programs. Hence, the intensity of exercise can be correlated with the rate of energy expenditure and with an estimated rate of fat metabolism. The presence of fat metabolism can be used to increase the number of points awarded for the exercise. For example, the number of points awarded for a given caloric expenditure can be increased by a certain percentage, for example 20%, for activity intensities known to be significant in inducing fat burning processes. Fat metabolism and metabolic rate can be monitored for time periods after the completion of an exercise routine, so as to allow the determination of the long term effects of an exercise on metabolic processes and fat burning. These measurements can be used in creating a physiological model for the person, by which the effect of exercise can be estimated more accurately.

Furthermore, using the indirect calorimeter disclosed in U.S. patent application Ser. No. 09/630,398, the anaerobic threshold can be determined from the measured respiratory quotient, the ventilatory equivalent for oxygen, and/or the ventilatory equivalent for carbon dioxide. For some exercise programs, exercising close to or above the anaerobic threshold is preferred, as this can provides certain physiological benefits. In this case, additional activity points can be provided for activity in the recommended activity zone.

A device can be provided which monitors the ketone production during an exercise, and provides feedback to the person to encourage them to continue exercising when fat metabolism is detected. Also, an activity sensor can be calibrated using a ketone detector, so that during future exercises the intensity level required for fat metabolism can be indicated to the person without the need to use a ketone sensor.

For different exercise conditions, points per unit time can be scaled according to appropriate equations. For example, the additional energy expenditure due to treadmill use is often stated to be proportional to treadmill speed. Hence, if a certain point value is achieved in fifteen minutes at two mph, it can be assumed that twice that point value is achieved for twice the treadmill speed.

The points per unit time, or per exercise repetition, or per other unit of exercise, can be established for a variety of exercises, such as cycling, running, running on the spot, jogging, walking, swimming, skiing, and the like. The activity point expenditure can be adjusted according to speed, number of repetitions, exercise intensity, distance, or other appropriate activity level parameter.

Improved Exercise System

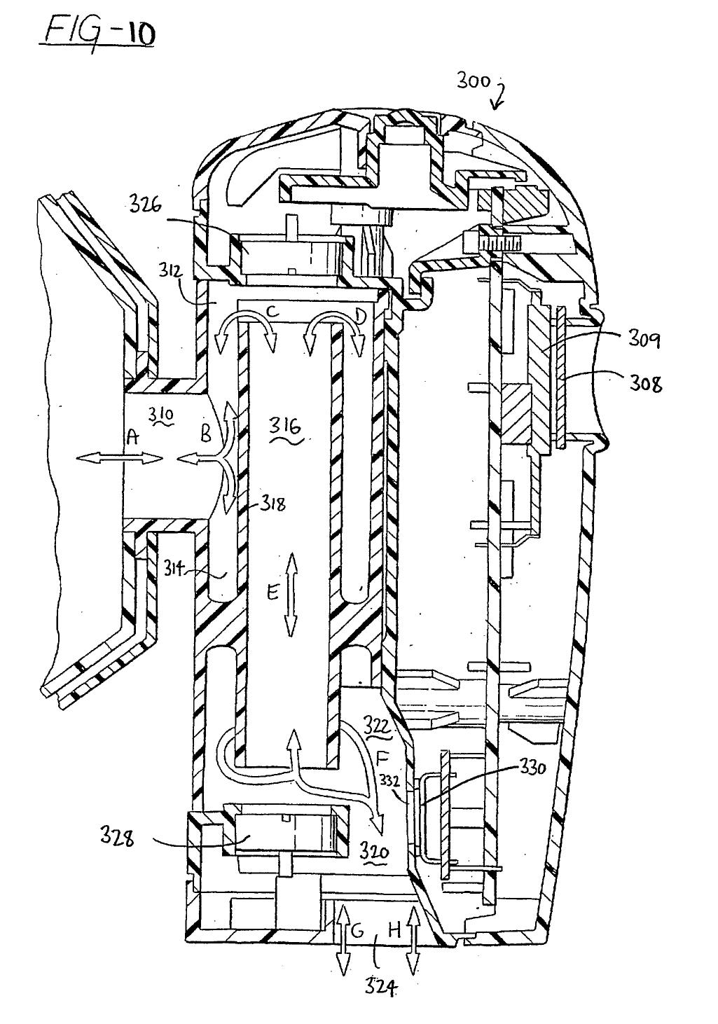

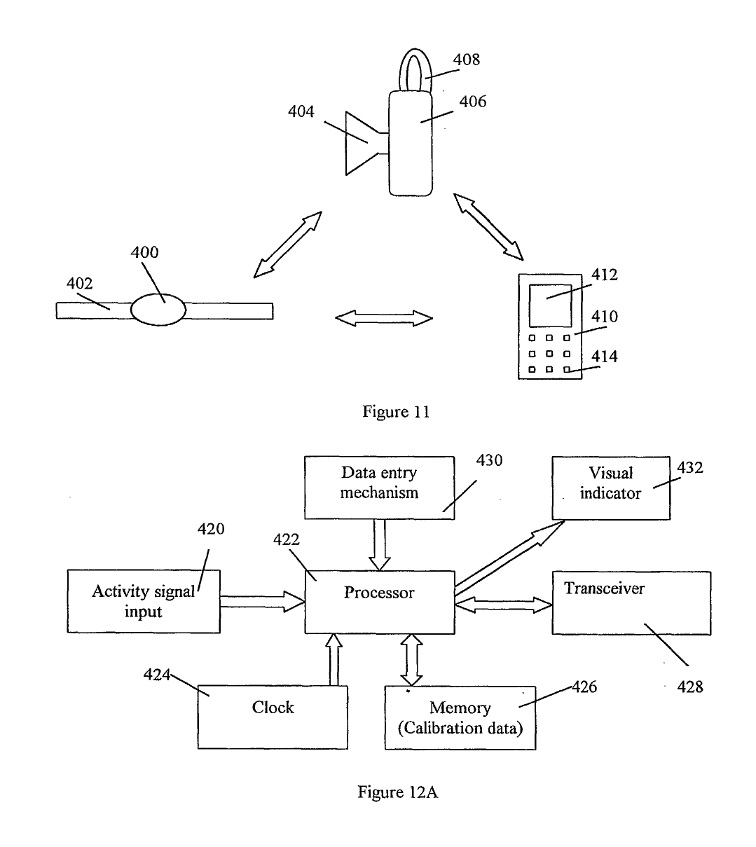

FIG. 11 shows an improved physical activity monitoring system. The system comprises an activity monitor 400 and a belt 402, used to secure the activity monitor to the body of the person through encircling the person's body, wrist or the like. A combined indirect calorimeter and ketone sensor 406, having a mask 404 and straps 408 for securing the device to the person's head. A portable computing device 410 has a display 412 and a data entry mechanism 414.

The person wears the activity monitor 400 on belt 402 during an exercise program.

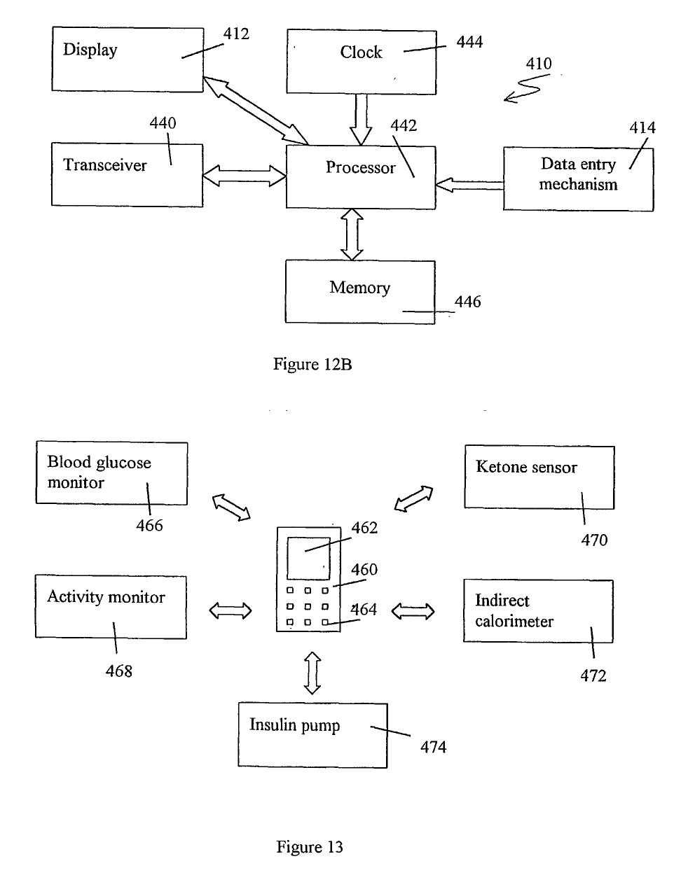

FIG. 12A shows a schematic of a possible embodiment of the activity monitor 400, comprising an activity sensor 420, a processor 422, a clock 424, a memory 426, a transceiver 428, a data entry mechanism 430, and a visual indicator 432. The data entry mechanism can be a button pressed to indicate the start and end of an exercise, or a numeric keypad to enter calibration data. The visual indicator can be a lamp, such as a light emitting diode, to indicate operation of the device. A number of lamps can further be used to provide feedback related to the energy expended during the exercise.

FIG. 12B is a schematic of a possible embodiment of a portable computing device, or other device having a display capabilities, used in system embodiments of the present invention. The portable computing device 410 has display 412 and a data entry mechanism 414 (as shown in FIG. 11), and further comprises a transceiver 440, a processor 442, a clock 444, and a memory 446.

The combined indirect calorimeter and ketone sensor 406 comprises a 0metabolic rate meter, and a ketone sensor, and data analysis functionality so as to determine the metabolic rate (or oxygen consumption) of the person, along with a determination of the person's breath ketone concentration.

The activity sensor provides a signal correlated with the physical activity of the person, and can take many forms, for example as disclosed in related co-pending U.S. Prov. Apps. Nos. 60/225,101 (filed Aug. 14, 2000), 60/225,454 (filed Aug. 15, 2000) and 60/228,680 (filed Aug. 29, 2000). In this example, the activity sensor is a pedometer which provides a signal correlated with repetitive motion, such as running on the spot.

A software application program on the portable electronic device can be used to guide the person through an exercise routine. For example, a person can be instructed to run on the spot at an increasing rate. In this example, the rate of the exercise corresponds to the frequency of running motion. During a calibration process, the person breathes through the combined indirect calorimeter/ketone sensor during the exercise program. The person runs on the spot at an increasing rate. The metabolic rate is correlated with the exercise rate, the signal from the metabolic rate meter (indirect calorimeter), and with the signal from the ketone sensor. At some exercise intensity or exercise rate, or after some time at a given activity level, the ketone detector will indicate fat burning. Embodiments of the indirect calorimeter can also provide measurement of respiratory quotient during exercise. The respiratory quotient can be used in addition to or instead of the ketone detection so as to provide indication of fat metabolism. For example, the person may have to exercise for a certain time at a given exercise intensity, so as to induce significant fat metabolism. Alternatively, fat metabolism may occur at a certain time during a periodically increasing intensity exercise program, or it may occur at some discrete intensity level if intensity levels are stepped in intensity. The purpose of the calibration procedure is to correlate the exercise rate with metabolic rate, and also to correlate the exercise rate with the nature of the metabolic processes within the person, in particular with the onset of significant fat metabolism. Future repetition of the exercise programs does not require the use of the combined indirect calorimeter/ketone detector. Either the exercise rate or signal from the activity monitor can be used to estimate the metabolic rate of the person during the exercise, and the metabolic processes occurring within the person during that exercise program. The electronic device 410 receives signals from the activity monitor 400 preferably over a wireless communication link. However, a cable connection can also be used. The electronic device 410 can be used to store data from the calibration process discussed above, and can in the future indicate a rate of activity energy expenditure and fat burning to the person, through storage of calibration data in the memory, and the reception of an exercise rate or activity monitor signal, for example by receiving transmissions using the transceiver, or through manual input of exercise rate through the data entry mechanism. Further, the electronic device can be used to indicate the level of fat burning, as determined from calibration processes described above. Calibration data can be transmitted from electronic device 410 to activity sensor 400, for example using a wireless protocol, allowing the visual indicator of the activity monitor to reflect calories expended and the onset of fat burning metabolic processes.

The combined indirect calorimeter and ketone sensor can be used in all exercise programs to indicate energy expenditure and the onset of fat burning. However, the calibration of an activity sensor allows an unobtrusive sensor to be worn during activities, such as walking, running, swimming, and other sports and exercise activities.

A number of physiological parameters can be correlated with fat burning, for example as listed in related co-pending U.S. Prov. App. No. 60/228,680 (filed Aug. 29, 2000) These physiological parameters include heart rate, body temperature, respiration rate, respiration volume, and the like. Other physical parameters can also be used, such as speed as determined by a positioning system such as a global positioning system (GPS). These physiological and other physical parameters can be combined as convenient, and used to predict or estimate the activity energy expenditure and metabolic processes within a person during an exercise program.

Hence, a person can receive feedback during an exercise, for example encouragement to continue an exercise if the person is known to be close to an exercise duration corresponding to significant fat burning. The person's metabolic rate and rate of fat burning can be monitored after the completion of an exercise, so as to determine the time dependence of metabolic rate and fat burning after an exercise is complete. This can be used to provide more accurate information within a calorie balance program.

Use of Ketone Sensor in Diagnosing Type 1 Diabetes

Embodiments of the invention described herein are useful for differentiating Type 1 and Type 2 diabetes, and for allowing improved diet control for a person suffering from Type 1 diabetes.

FIG. 13 illustrates diet and exercise control system for a person suffering from diabetes. The person carries a personal digital assistant (PDA) 460, having a display 462 and data entry mechanism 464, and has a glucose sensor 466 transmitting blood glucose levels to the PDA 460 using a wireless transmission protocol such as Bluetooth. The system further comprises an activity monitor 468, a respiratory ketone sensor 470, an indirect calorimeter 472, and an insulin pump 474. The insulin pump is omitted unless the person gains some advantage from insulin injections. Dietary intake can be entered into the PDA, using calorie management software advantageously adapted from, for example, the diet log software disclosed by Williams in U.S. Pat. Nos. 5,704,350 and 4,891,756. The double headed arrows represent communication links, preferably using a wireless protocol such as Bluetooth, IEEE802.11(b), wireless Ethernet, IR, optical links, or some other method. The ketone sensor is used to detect the onset of ketoacidosis. The calorie management software is adapted to receive a metabolic rate measurement from the indirect calorimeter, and an activity signal correlated with the physical activity level of the person from the activity monitor. The calorie management software is adapted to provide an alert if an onset of ketoacidosis is possible, for example due to a certain time period from eating, low levels of eating relative to calorie needs for resting metabolism and activity, high levels of activity such as due to exercise. An alert can be provided to the person at intervals to recommend ketone sensor usage, blood sugar measurements, other diagnostic procedure, or the administration of medications. The alert intervals can be correlated with metabolic rate, diet logging, activity, or other physiological parameters. The PDA can be in communication with a communications network, such as the Internet, preferably through a wireless communications link. Data can be transmitted to a remote computer for storage, analysis, and review by a physician or computer expert system. Feedback can be provided to the person over the communications network and be viewed on display 462. The feedback may comprise diet, exercise, and nutrition related advice, medical treatment suggestions, and electronic prescriptions. The electronic prescriptions can be simultaneously transmitted to a pharmacy, allowing the person to collect a medication, or the person can take the PDA with prescription information to a pharmacy, or the person can retransmit the electronic prescription to a pharmacy of their own choice over the communications network.

The PDA is used to track dietary intake and blood sugar levels, estimate possible future deviations of blood sugar from an acceptable range, and provide warnings and advice to the person. An indirect calorimeter, which can comprise the functionality of a ketone sensor, is used to determine the metabolic rate of the person. An activity sensor is used to provide a signal correlated with physical activity. These data are transmitted to the PDA, preferably using Bluetooth. Breath ketone sensing is used to detect the onset of the dangerous condition of ketoacidosis. Other portable electronic devices can be used in place of the PDA, such as other portable computing devices, wireless phones, pagers, e-books, tablet computers, wrist-mounted devices (which may comprise the functionality of PDA and glucose sensor), and the like.

A system for warning a person of the onset of ketoacidosis comprises a portable computing device carried by the person, a blood glucose sensor, and a respiratory analyzer (which device functions of indirect calorimeter and respired volatile organics detector, in two way communication using wireless communication. Data may also be transferred to or from any device using non-volatile memory cards, or via a wire.

The PDA and respiratory analyzer may be combined into a portable unitary device, or the respiratory sensors may be attached to the PDA for use. Also, the ketone sensing device may be combined or be separate from the calorimeter.

Other Geometries for Breath Analyzer

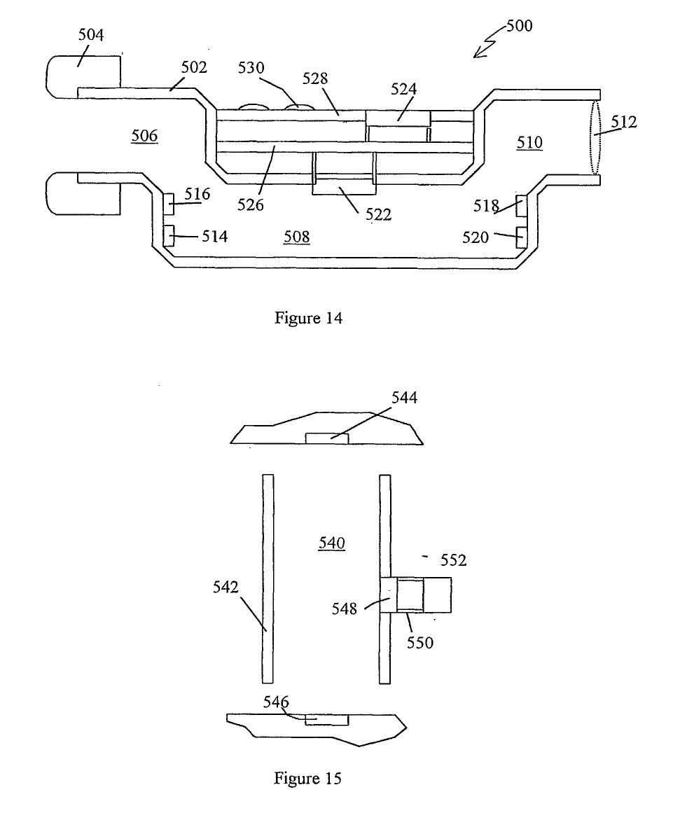

FIG. 14 illustrates another geometry for a breath analyzer with ketone detection capabilities. The respiratory analyzer device shown generally at 500 comprises a flow path housing 502, a mouthpiece 504 serving as an inlet conduit in fluid communication with inlet tube 506, a main flow path 508, outlet tube 510, and exhaust opening 512. (The nomenclature reflects a use for exhalation analysis, but inhalation can also be monitored, in which case the flow direction is reversed). The device has a pair of ultrasonic transducers 516 and 518, which act cooperatively as a flow sensor using techniques known in the art. For example, the techniques described by Delsing can be used, as described in U.S. Pat. No. 5,932,812; also those described by Harnoncourt in U.S. Pat. Nos. 5,647,370, 5,645,071, 5,503,151, and 5,419,326, incorporated herein by reference, can be advantageously used. The device further comprises a radiation emitter 514 and radiation detector 520, acting as an analysis system so as to detect ketones of exhaled breath. A fluorescence oxygen sensor 522 is also disposed on the inner surface of housing 502 to provide a signal correlated with a oxygen component concentration. The fluorescence oxygen sensor is mounted by pins on a circuit board 526, on which analysis circuitry and display 524 are also mounted. Finger grips 530 are disposed on the outer surface of an analysis housing 528, which encloses the circuit board 526 in conjunction with flow path housing 502. The shape of the housing is not critical, but preferably it is adapted to be held by one hand. The flow path for respiration preferably has a circular cross section, but can also be oval or other shape.

A fluorescence sensor can also combined with the coaxial geometries described above. FIG. 15 shows part of a respiratory analyzer having a coaxial geometry, comprising central flow path 540, flow tube 542, a pair of ultrasonic transducers 544 and 564, and a fluorescence ketone sensor 548 with connection 550 to fluorescence analysis system 552. The fluorescence analysis system may comprise the circuit disclosed in U.S. patent application Ser. No. 09/630,398, adapted to provide an electrical signal correlated with ketone concentration. The ultrasonic transducers 544 and 546 are used to determine the flow rate through the central flow path 540, using, for example, the methods described in U.S. patent application Ser. No. 09/630,398 to Mault et al. The fluorescence ketone sensor 548 is disposed on the inside surface of the flow tube 542, or elsewhere within the respiratory analyzer, instead of or in addition to other fluorescence gas sensors (such as oxygen sensors, carbon dioxide sensors), so as to provide a signal correlated with ketone levels in exhaled air.

The ketone signal, correlated with ketone concentration in the exhaled breath, can be integrated with a flow rate signal obtained from a flow sensor so as to determine the volume of ketones exhaled. Alternatively, the ultrasonic transducers can be omitted, with only the average concentration or some other concentration parameter of exhaled ketones determined. In other embodiments, the density of exhaled gas and hence carbon dioxide concentration can be determined as disclosed in WO 00/07498, allowing a metabolic rate to be determined using the device along with ketone concentration.

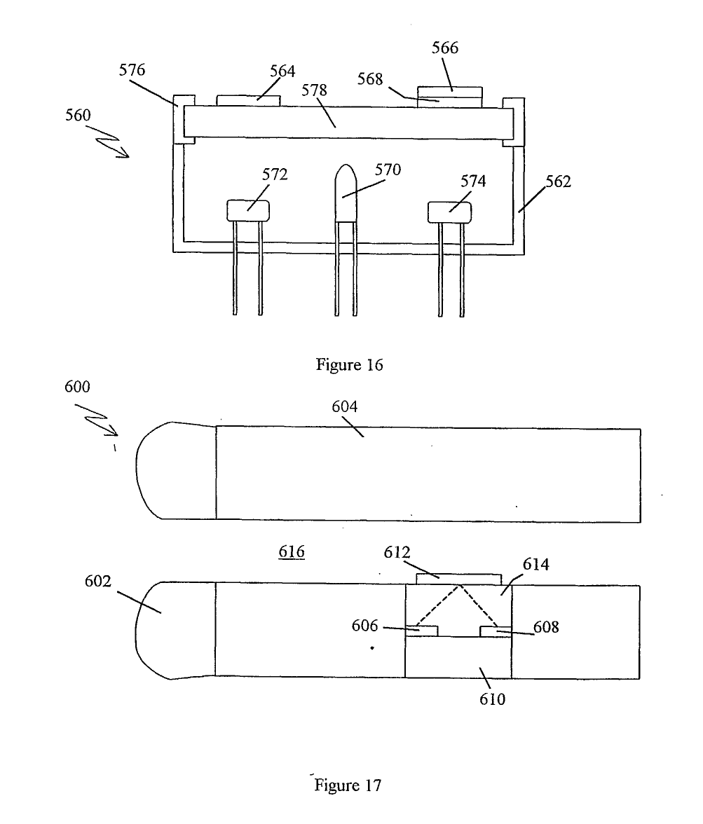

FIG. 16 shows a possible embodiment of a fluorescence ketone sensor. The sensor shown generally at 560 comprises a housing 562, a sensor fluorescence film 564, a ketone impermeable membrane 566, a reference fluorescence film 568, a light emitting diode 570, a sensor photo-detector 572, and a reference photo-detector 574. The fluorescence films 564 and 568 fluoresce in response to irradiation by the light emitting diode 570. The reference film is exposed to ketones in exhaled breath, so that the fluorescence intensity reduces (due to fluorescence quenching) in a manner correlated with the concentration of ketones. The reference fluorescence film 568 is separated from exhaled breath by the impermeable membrane 566, and so provides a reference signal unaffected by ketone levels in exhaled breath, but subject to the same environmental conditions as the sensor film. The fluorescence films 564 and 568 are deposited on substrate 578, which slides into a horizontal slot in the housing 576. The substrate 578 can be removed for calibration or replacement if necessary (for example due to photodegradation of the fluorescence films). Other fluorescence sensor embodiments can be advantageously adapted from those disclosed in Int. App. WO00/13003, WO99/46600, WO98/52023, WO98/52024, WO92/15876, and U.S. Pat. Nos. 5,517,313; 5,894,351; 5,917,605; 5,910,661, all to Colvin, the contents of which are incorporated herein by reference. The fluorescence film may comprise a transition metal compound, such as a ruthenium complex.

Device with Straight Flow Path

FIG. 17 shows a respiratory analyzer 600, comprising mouthpiece 602, cylindrical housing 604, radiation emitter 606, radiation detector 608, transparent substrate 614, and analysis module 610, which may comprise a Peltier cooler (or other cooler, such as a water cooler) to cool substrate 614. A person holds the device to their mouth, and breathes through mouthpiece 602, so that exhaled air is conveyed along flow path 616. The exhaled air may be dried by conventional means, e.g. using silica gel. Volatile organic compounds such as acetone are condensed as a film 612 on a cooled radiation-transparent substrate 614. Radiation from radiation emitter 606 is reflected from the condensed film 612, or from the inside surface of substrate 614, and directed to radiation detector 608. For example, attenuated total reflection IR spectroscopy can be used to analyze the film 612. The film 612 can absorbs IR radiation at selective wavelength and causes attenuation of the reflection, which is detected by analyzing the signal from the detector. An optical filter may also be placed in the path of the radiation, in front of the detector, to pass only the wavelength range(s) of interest, e.g. near the carbonyl stretch vibrational frequency. In other embodiments, the cooler can be omitted. The film 612 can be a film providing a calorimetric response to the presence of an exhalation component, such as ketones. The calorimetric response can be detected from change in optical reflection characteristics.

In other embodiments, a calorimetric indicating film is disposed at some location within the flow path, so as to provide an indication of ketones within the breath. Colorimetric chemistry sensitive to ketones are described by Kundu et al. in U.S. Pat. No. ______ and De Castro in U.S. Pat. No. 5,834,626. These, or other colorimetric indicating films, sensor patches, and the like, can be incorporated into a mask, mouthpiece, other respiratory connector, or at some location within the flow path, so as to provide indication of ketones in the breath after, for example, several minutes of respiration, such as during a metabolic rate measurement using an indirect calorimeter. Conventional colorimetric indicating methods provide a sampling method for collecting exhaled breath, however in the improved colorimetric detection method described here no sampling or collection is necessary, as the person breathes over the calorimetric indicator film for a certain duration.

Other Laser Detection Methods

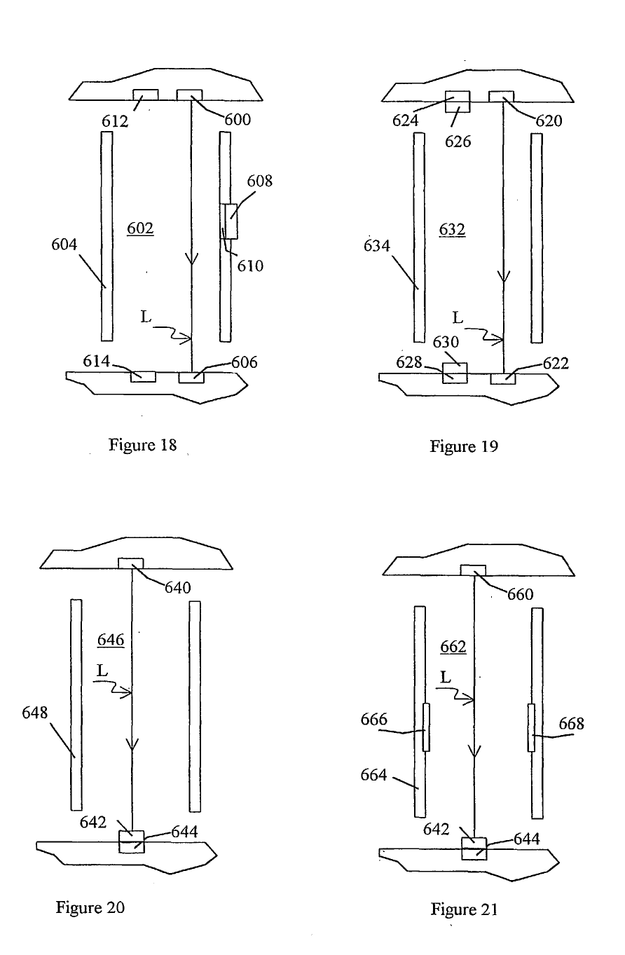

The coaxial flow path is well suited to laser-based detection methods. FIG. 18 shows a laser source 600, producing a laser beam L propagating along the flow path 602 bounded by flow tube 604. The laser is absorbed by laser absorber 606. The laser source can be used to excite atoms or molecular species in the flow column. For example, the laser 600 may be designed with an emission wavelength so as to induce photoexcitation of acetone. Fluorescence from the excited molecules is sensed by a detector 608. A filter 610 is placed in front of the detector 608, so as to pass fluorescence to the detector, while rejecting other wavelengths (particularly the laser wavelength). Fluorescence or phosphorescence of excited molecules may be detected, preferably in the IR or optical regions of the electromagnetic spectrum. IR emission from laser-excited molecules may be detected in some embodiments. Ultrasonic transducers 612 and 614 are used as a flow sensor, as is known in the art, and can for example be used to detect the start of an exhaled breath so as to initiate detection of an exhaled breath component.

The laser source is preferably a solid state laser such as a semiconductor laser, but may also be a light-emitting diode or other electroluminescent source. The emission wavelength is preferably in the near-IR or visible regions of the spectrum, but may also be mid-IR, far-IR, UV, or elsewhere in the electromagnetic spectrum. Microwave radiation combined with magnetic fields in principle allows electron spin resonance detection of NO, though sensitivity will be low. The laser emission wavelengths are chosen for detection selectivity and sensitivity. The laser beam may undergo multiple reflections backwards and forwards through the flow column for increased levels of photoexcitation, fluorescence, or other factors increasing sensitivity, e.g. by replacing absorber 606 by a reflector. The laser emission may be modulated, with phase sensitive detection used for enhanced sensitivity. The laser emission may be polarized, and polarizers may be placed in front of the detector.

The configuration shown in FIG. 18 is also suitable for Raman detection of molecules within the flow column. In this case, a narrow band filter or dispersive element is placed in front of the detector 608 so that only Raman scattered light may reach the detector 608. In other embodiments, a fluorescence detector can be used to detect photoexcited molecules, for example using the interaction of a fluorescent transition metal compound with photoexcited molecules.

FIG. 19 shows both backwards and forwards scattering/fluorescence detection schemes. Laser radiation L from laser source 620 is absorbed by laser absorber 622. In back-scattering detection, a detector 624 adjacent to the laser 620 is used to detect fluorescence or scattered laser radiation. A filter 626 is used to transmit only radiation of interest to the detector. In this, and all shown schemes, polarized laser radiation and/or polarized detection may be used, along with reflection of the laser beam to increase path lengths through the flow column. In the forward scattering/fluorescence geometry, the detector 628 and appropriate filter 630 are at the opposite end (to the laser) of the flow path 632 formed by flow tube 634.

FIG. 20 shows a scheme in which a narrow band filter 642 is used to filter out (absorb or selectively reflect back) radiation from laser 640, allowing fluorescence or scattered radiation to pass through to the detector 644. Gas components in the flow column can also be detected using radiation absorption, e.g. if the filter 642 passes laser radiation to the detector 644. IR absorption is particularly useful for identifying carbon dioxide, ketones, and aldehydes using the fundamental or overtone absorption of the carbonyl group. Respiration components of exhaled air passing along flow path 646 formed by flow tube 648 can be determined.

Photoacoustic and Ultrasonic Detection

The photoacoustic effect can be used to detect respiratory components. In U.S. Pat. No. 5,616,826, Pellaux et al. describe a photoacoustic detection system for nitric oxide (NO). Referring to FIG. 18, laser source 600 is modulated to produce laser pulses, at an appropriate wavelength depending on the gas component to be detected. At least one microphone is used to detect the photoacoustic signal. Suitable microphones can be incorporated by micromachining technology into micromachined ultrasonic transducers 612 and 614.

Micromachined ultrasonic transducers may be used to investigate the ultrasonic spectra of respired gases over broad frequency ranges, e.g. 50 kHz-10 MHz. Resonances due to individual molecular species may be detected and used to determine the concentration of that species in the respired gases.

Photoionization Detection

FIG. 21 shows a scheme in which selective photoionization of a chosen molecular or atomic species is used for detection. The emission wavelength of laser 660 is selected so that only the required analyte is photoionized. The laser beam L passes along the flow path 662 formed by flow tube 664. (The direction of the beam can be at any angle to the direction of exhalates along the flow path, and can undergo reflection). A high voltage is applied between two electrodes 666 and 668, mounted on the flow tube 664. The current detected flowing across from one electrode to the other is correlated to the concentration of ionized species, hence the presence and concentration of that species can be determined. A detector 644 with filter 642 can be used to monitor fluorescence, absorption, or scattering, for example as discussed above in relation to FIG. 20.

Electric discharges within the flow path may also be used for selective ionization of molecules or radicals.

Mass spectroscopy is generally accepted in the art to be an expensive analytical tool, well outside of the realm of consumer appliances. However, if the species to be detected is known, a system configuration of e.g. laser photoionization, electric and/or magnetic fields, and ion detecting apparatus can be designed specifically to detect an analyte of known mass/change ratio. Such a simplified configuration would be considerably less expensive than a conventional instrument, and suitable for respiratory analysis of acetone. For example, a conventional mass spectrometer can be advantageously modified to provide a signal correlated with the concentration of ions having the mass/charge ratio of singly (or multiply) charged acetone, and used with a respiratory analyzer, indirect calorimeter, and the like. Conventional mass spectrometers can also be used with the GEM or other indirect calorimeter, e.g. by connecting the gas inlet of the spectrometer to the source/sink of respired gases.

Micromachined Sensors

In U.S. Pat. Nos. 6,050,722; 6,016,686; 5,918,263; 5,719,324; 5,445,008, and related applications, Thundat and co-inventors describe micro-mechanical sensors which may be adapted for NO or other respiration component detection, e.g. through the change of resonance frequency of a micromechanical structure due to gas adsorption on the surface (alternatively absorption, chemisorption, physisorption, etc., on or in the surface). Micromechanical sensors may also be used in temperature sensing (for example as described in U.S. Pat. No. 6,050,722). Micromachined sensors may be placed along the flow column to detect trace respiration components. Micromachined devices may also be advantageously fabricated containing some combination of ultrasonic transducers, pressure sensors, humidity sensors, trace gas sensors, and temperature sensors, which may be useful in respiration analysis.

There may be small quantities of glucose in exhaled breath. These quantities are related to blood glucose levels, due to processes e.g. within the lungs. Hence, it would be useful to include a glucose sensor in the flow path. This may be a fluorescence sensor, calorimetric sensor, micromechanical sensor, or other sensor technology e.g. using enzymes such as glucose oxidase. For example, a micromachined sensor may have a surface coated with glucose-binding chemistry.

In other embodiments a blood ketone sensor can be used to provide a ketone signal, correlated with ketone body levels in the blood and ketone concentrations in exhaled breath. Interstitial fluid can also be analyzed to provide a ketone signal, for example using a microcapillary system as described in U.S. Provisional App. No. 60/257,138. A sensor providing a signal responsive to ketone bodies can be used in place of or in addition to a glucose sensor.

An improved diet management program for a person can comprise: providing an indirect calorimeter; providing a ketone sensor; providing a calorie management device (such as a PDA having software for diet logging, activity logging, resting energy expenditure recording, and calorie balance calculation), monitoring calorie balance, resting metabolic rate, and ketone concentration in exhaled breath, and providing feedback to the person based on the monitored parameters. The presence of ketones in the breath is associated with the person running a calorie deficit (expending more calories through resting energy expenditure and activity energy expenditure than consumed through diet). Hence, if the person indicates a sustained calorie deficit, but ketones are not present in the breath, the person may be under-reporting diet, and the person can be provided with feedback on improved diet logging methods. Aims of the improved program can include sustaining a certain value of resting energy expenditure, and sustaining a minimum concentration of ketones in the breath (which is indicative of fat burning).

The invention is not to be limited by the examples described above.

Having described my invention, I claim:

1. A respiratory analyzer for a person, the person having a metabolic rate and blood, comprising:
a flow path, through which the person breathes;
a metabolic rate meter, providing metabolic data correlated with the metabolic rate of the person;
a sensor, providing a ketone signal correlated with a concentration of a respiratory component in exhalations of the person, wherein the concentration of the respiratory component is correlated with a level of ketone bodies in the blood of the person;
a display;
an electronic circuit, receiving the ketone signal and the metabolic data, and providing a visual indication of the metabolic rate and the ketone signal on the display.

2. The respiratory analyzer of claim 1, wherein the metabolic rate meter comprises a pair of ultrasonic transducers.

3. The respiratory analyzer of claim 2, wherein the metabolic rate meter further comprises an oxygen sensor.

4. The respiratory analyzer of claim 2, wherein the metabolic rate meter further comprises a carbon dioxide sensor.

5. The respiratory analyzer of claim 1, wherein the ketone sensor comprises a radiation emitter and a radiation detector, and wherein radiation emitted by the radiation emitter passes through a part of the flow path.

6. The respiratory analyzer of claim 1, wherein the ketone sensor comprises a fluorescence film, wherein a fluorescence intensity of the fluorescence film is correlated with the concentration of the respiratory component.

7. A respiratory analyzer, comprising:
a flow path operable to receive and pass exhaled gases, the flow path having a first end in fluid communication with a respiratory connector and a second end in fluid communication with a source and sink for respiratory gases, the respiratory connector configured to be supported in contact with a subject so as to pass exhaled gases as the subject breathes, the flow path comprising a flow tube through which the exhaled gases pass, and a chamber disposed between the flow tube and the first end, the chamber being a concentric chamber surrounding one end of the flow tube; and
a ketone sensor, providing a ketone signal correlated with a ketone concentration in exhaled breath passing through the flow path.

8. The respiratory analyzer of claim 7, wherein the ketone sensor comprises: a radiation emitter and a radiation detector, wherein radiation emitted by the radiation emitter passes through a part of the flow path and is detected by the radiation detector.

9. The respiratory analyzer of claim 7, wherein the ketone sensor comprises a fluorescent film, wherein a fluorescence intensity of the fluorescence film is correlated with the ketone concentration in the flow path.

10. The respiratory analyzer of claim 7, further comprising a flow rate sensor.

11. The respiratory analyzer of claim 10, wherein the flow rate sensor comprises a pair of ultrasonic transducers.

12. An improved method for exercise management for a person performing an activity, the method comprising:
providing an activity monitor;
providing a metabolic rate meter;
providing a respiratory analyzer having a ketone sensor;
monitoring an activity signal from the activity monitor;
monitoring a metabolic rate signal from the metabolic rate meter, wherein the metabolic rate signal is correlated with a metabolic rate of the person;
monitoring a ketone signal from the ketone sensor, wherein the ketone signal is correlated with a concentration of ketone bodies in blood of the person; and
correlating the activity signal with the metabolic rate signal and the ketone signal; whereby the activity signal can then be used to determine a metabolic rate and an estimate of fat burning for the person during an exercise, allowing improved exercise management.

13. The method of claim 12, wherein a unitary device comprises the metabolic rate meter and the ketone sensor.
